(19) United States
(12) Patent Application Publication
(10) Pub. No.: US 2016/0015457 A1
(43) Pub. Date: Jan. 21, 2016

(54) Multi-Mode Storage Apparatus

(71) Applicant: Richard Hurst, Vero Beach, FL (US)

(72) Inventor: Richard Hurst, Vero Beach, FL (US)

(21) Appl. No.: 14/335,600

(22) Filed: Jul. 18, 2014

(51) Int. Cl. A61B 19/02

(52) U.S. Cl. CPC A61B 19/0271; A61B 2019/0277; A61B 2019/0278

(57) ABSTRACT

The invention is a new horizontal syringe storage system for larger syringes which provides a high degree of product safety, storage and accessibility. The horizontal syringe storage system defines offset scallops to provide for tray nesting when stacked in a similar direction and for tray stacking when an upper tray is rotated 180° relative to a lower tray. In use, the upper trays do not touch product in a lower tray. For one embodiment, the syringe storage system comprises raised nubs for vertical alignment between stacked trays. Such raised nubs of a lower tray operably cooperative with the scallop pockets of an overhead tray. This simple feature aligns trays on two axes and limits tray slippage, essentially offering unlimited syringe tray stacking.

### CLAIM TO PRIORITY

This application claims priority to and is a continuation in part of U.S. patent application Ser. No. 29/487,172 filed on 6 Apr. 2014, by inventor Hurst, wherein such application is incorporated by this reference for all that it discloses for all purposes.

### FIELD OF THE INVENTION

This invention relates in general to the field of material handling equipment and in particular to a medical material handling apparatus and more particularly to the construction of a syringe storage apparatus for primary use in the medical field.

### BACKGROUND OF THE INVENTION

Space is a major issue in all companies and in particular pharmaceutical companies. For example, to store and transport horizontal syringe storage trays (syringe trays) in the smallest space possible, it is important that such syringe storage trays NEST when not filled so that storing empty trays requires minimal cubic footage of space. However, when such syringe storage trays are being used to store syringe systems, such syringe storage trays should be stackable so that they can sit on top of one another where the syringes of a lower tray remain untouched by an upper tray. Further, when vertical stacking is used the syringe storage tray should be crush proof when such trays are stacked to a predefined height (i.e. under normal use).

Thus, what is needed, for at least syringe systems, is a multi-mode storage apparatus.

Notably, in a commonly owned U.S. Pat. No. 7,766,164 issued Aug. 3, 2010, (incorporated herein by this reference for all that it teaches for all purposes), the current inventor, Hurst, teaches a medical tray and cover apparatus. The apparatus comprises a tray body having an open top and an open front with a removable cover that covers the tray top and front. Such trays are transparent so that its contents are visible without removing the cover. The medical trays are stackable with the cover in place and lockable such that the ability to stack the trays is not interfered with.

Hurst has now developed a revolutionary syringe storage apparatus (where some embodiments resemble a “flat tray”). Such syringe storage apparatus can be used in a stand-alone configuration or, if suitably sized, such syringe storage apparatuses can be stacked and stored in the above identified U.S. Pat. No. 7,766,164 Medical Tray and Cover Apparatus.

The new Hurst multi-mode storage apparatus provides the above identified features and additional features that are provided by combining the various improvements as described below.

### SUMMARY OF THE INVENTION

Some of the objects and advantages of the invention will now be set forth in the following description, while other objects and advantages of the invention may be obvious from the description, or may be learned through practice of the invention.

Broadly speaking, a principle object of the present invention is to provide a multi-mode storage apparatus that requires minimal storage volume.

Another principle object of the present invention is to provide a syringe storage apparatus that requires minimal storage volume by providing a first stacking mode when being used to store a syringe system and a second nesting mode when not storing a syringe system.

Yet another principle object of the invention is to provide a syringe storage apparatus that requires minimal storage volume by providing a first stacking mode when being used to store a syringe system and a second nesting mode when not storing a syringe system and further providing a movement limiter when the syringe storage trays are being used in the first stacking mode.

An additional principle object of the invention is to provide a syringe storage apparatus that requires minimal storage volume by providing a first stacking mode when being used to store a syringe system and a second nesting mode when not storing a syringe system and further providing a crush proof construction when a plurality of apparatuses are in the stacking mode (when used as intended).

One embodiment of a multi-mode storage tray comprises a body defining a pair of opposed surfaces where one surface is a top surface and the other surface is a bottom surface. The distance between such surfaces defines the body thickness.

The opposed surfaces further define a plurality of side portions comprising a first side-portion defined along a first side of the top surface, a second side-portion defined along a second side of the top surface, a third side-portion defined along a third side of the top surface, and a fourth side-portion defined along the fourth side of the top surface. Such side portions extend away from the top surface to define the body's height (or depth if you will). Preferably at least two opposing sides extend out to the same distance so that the tray sits level on a level surface.

The body further defines four corners where the side portions meet. More specifically, a first end of the first side-portion meets a first end of a second side portion to define a first body-corner. Similarly, a second end of the second side-portion meets the first end of a third side portion to define a second body-corner. Likewise the second end of said third side-portion meets the first end of a fourth side portion to define a third body-corner. Finally, the second end of the forth side-portion meets the second end of the first side portion to define a forth body-corner. Preferably, the perimeter of the body defines a rectangular shape.

The top surface defines a plurality of channels in horizontal alignment with at least one side-portion where adjacent channels are separated by a hollow ridge. The plurality of channels and hollow ridges are surrounded by a border-region. Such border region defines a least one of (a) an open-top-border-region, (b) open-top-border-region surrounded by a hollow-top-border-region and (c) a hollow-top-border-region.

Notably, at least two opposing side-portions each define a plurality of side-recesses. One type of side-recess is a scallop shaped indention. The distance from a first side-recess to a first body-corner is different from the distance from a last side-recess to a second body-corner. Such feature provides for a tray-storage “nesting” mode and a syringe-storage mode when two or more syringe storage apparatuses are stacked on top of each other. More specifically, when two syringe storage apparatuses are oriented in the same alignment, the side-recess align and the syringe storage apparatus “nest” (the bottom syringe storage tray slides up into the upper tray thereby “nesting” thereby minimizing the height required to store same). When a top tray is oriented 180 degrees out of alignment compared to the bottom tray, the side-recesses do not line up and the top tray stacks on top of the bottom tray.

The channels are configured to receive a syringe system so that the upper most portion of the syringe system is lower than the upper most portion of said top surface when said syringe system is associated with a channel. Thus, when the syringe storage systems are associated with trays used in tray stacking mode, the syringe systems stored in a lower tray are not touched by the bottom of an upper tray.

For one embodiment the side-portions extend away from the top surface at an angle slightly greater than 90 degrees to enhance said tray-storage mode. For yet another embodiment, the top surface further defines a vertical-extension along its edge and disposed between adjacent side-recesses so that the vertical-extension associates with a side-recess of a second multi-mode storage tray when the two trays are stacked on top of each other and oriented in the tray stacking mode. Such a configuration retards sliding/movement between syringe storage apparatuses.

Additional objects and advantages of the present invention are set forth in the detailed description herein or will be apparent to those skilled in the art upon reviewing such detailed description. Also, it should be further appreciated that modifications and variations to the specifically illustrated, referenced, and discussed steps, or features hereof may be practiced in various uses and embodiments of this invention without departing from the spirit and scope thereof, by virtue of the present reference thereto. Such variations may include, but are not limited to, substitution of equivalent steps, referenced or discussed, and the functional, operational, or positional reversal of various features, steps, parts, or the like. Still further, it is to be understood that different embodiments, as well as different presently preferred embodiments, of this invention may include various combinations or configurations of presently disclosed features or elements, or their equivalents (including combinations of features or parts or configurations thereof not expressly shown in the figures or stated in the detailed description).

### BRIEF DESCRIPTION OF THE DRAWINGS

A full and enabling description of the present subject matter, including the best mode thereof, directed to one of ordinary skill in the art, is set forth in the specification, which makes reference to the appended figures, in which:

FIG. 1 is an elevated top perspective view of one exemplary storage apparatus in a first orientation;

FIG. 2 is an elevated top perspective view of one exemplary storage apparatus in a second orientation where such storage apparatus is rotated 180 degrees form the first orientation depicted in FIG. 1;

FIG. 3 is a close up view of the circled area depicted in FIG. 1;

FIG. 4 is a bottom plan view of the storage apparatus depicted in FIG. 1;

FIG. 5 is a top plan view of the storage apparatus depicted in FIG. 1;

FIG. 6 is a side elevational view from perspective A-A depicted in FIG. 5;

FIG. 7 is a first side elevational view of the storage apparatus depicted in FIG. 1;

FIG. 8 is a second side elevational view of the storage apparatus depicted in FIG. 1;

FIG. 9 is a top perspective view of a plurality of storage apparatuses in a first syringe-storage stacking mode;

FIG. 10 is a perspective elevated view of a syringe storage apparatus (10) associated with a tray-storage-device (13);

FIG. 11 is a perspective elevated view of one alternative embodiment of a storage apparatus comprising top-extensions;

FIG. 12 is a top plan view of two storage apparatuses oriented 180 degrees out of phase/alignment relative to each other;

FIG. 13 is a first side elevational view of the storage apparatus depicted in FIG. 11;

FIG. 14 is a second side elevational view of the storage apparatus depicted in FIG. 11;

FIG. 15 is an end elevational view of the storage apparatus depicted in FIG. 12;

FIG. 16 is an elevated perspective view of a plurality of storage apparatuses in a tray-storage stacking mode;

FIG. 17 is an elevated perspective view of a plurality of storage apparatuses in a syringe-storage stacking mode; and

FIG. 18 is an elevated perspective view of one exemplary embodiment of a storage apparatus cover.

Repeat use of reference characters throughout the present specification and appended drawings is intended to represent the same or analogous features or elements of the present technology. Various objects, advantages, and features of the invention will become apparent to those skilled in the art from the following discussion taken in conjunction with the drawings.

### DESCRIPTION OF THE PREFERRED EMBODIMENTS

Reference now will be made in detail to the embodiments of the invention, one or more examples of which are set forth below. Each example is provided by way of explanation of the invention, not limitation of the invention. Indeed, it will be apparent to those skilled in the art that various modifications and variations can be made in the present invention without departing from the scope or spirit of the invention. For instance, features illustrated or described as part of one embodiment can be used on another embodiment to yield a still further embodiment. Thus, it is intended that the present invention covers such modifications and variations as come within the scope of the appended claims and their equivalents. Other objects, features, and aspects of the present invention are disclosed in or may be determined from the following detailed description.

Repeat use of reference characters is intended to represent same or analogous features, elements or steps. It is to be understood by one of ordinary skill in the art that the present discussion is a description of exemplary embodiments only, and is not intended as limiting the broader aspects of the present invention.

For the purposes of this document two or more items are “mechanically associated” by bringing them together or into relationship with each other in any number of ways including a direct or indirect physical connections that are intended to be releasable (snaps, screws, bolts, buttons, zippers etc.), connections intended to be less releasable (rivets, welding etc.), and/or movable (rotating, pivoting, oscillating, etc.)

Further, while the examples used in this document relate generally to medical storage apparatuses/trays particularly well suited to store syringe systems, the disclosed storage apparatuses can be used to store any number of items.

Please note that this document contains headers that are intended for convenience only (general place locators) and are not to be used in interpreting the meaning of this document.

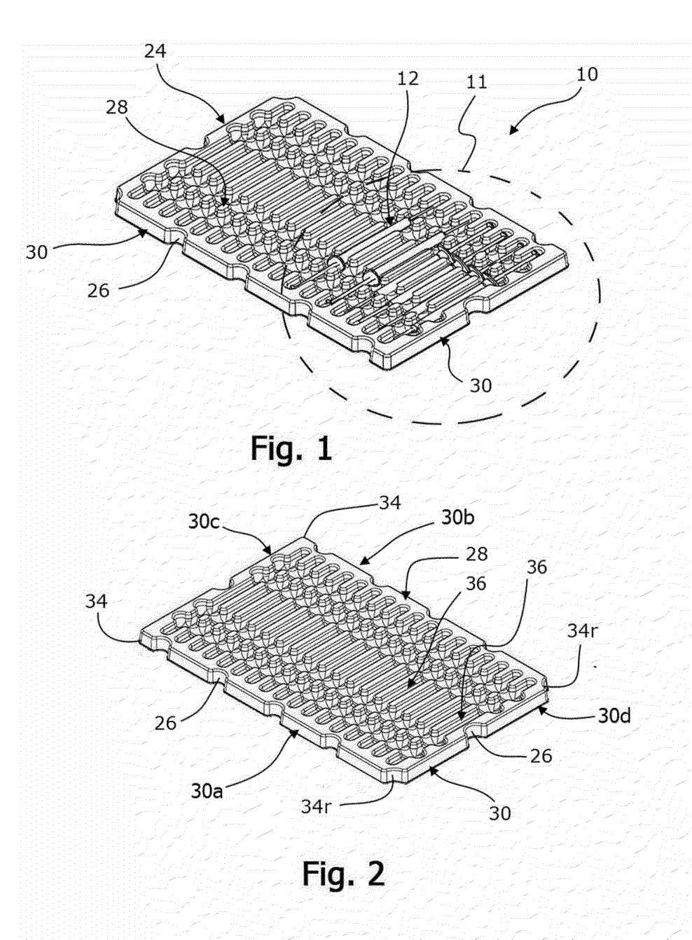

Referring now to FIG. 1, one exemplary embodiment of a storage apparatus (10) configured for storing a syringe system (12) is presented. Exemplary embodiments of a storage apparatus (10) include medical trays and multi-mode storage trays as described later. Storage apparatus (10) is preferably formed using a Federal Drug Administration (FDA) approved acrylonitrile butadiene styrene (ABS) material. That said, any suitable material may be used wherein such material preferably allows for the use of common sterilization processes and provides for a crushproof structural integrity when storage apparatus (10) is used as described herein.

### Syringe Systems

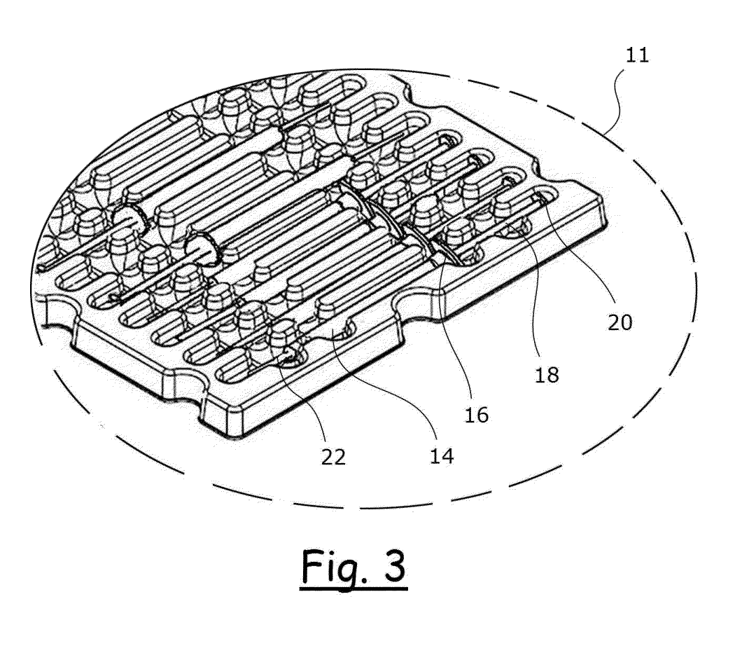

As best seen in FIG. 3, syringe systems (12) may be any type of system for holding a substance. Exemplary syringe systems (12) include prefilled syringes, empty syringes, syringes with and without flanges (16) , plungers (18) and cannulas. Storage apparatus (10) is particularly well suited for storing syringe systems having a barrel portion (14) configured to hold 1 milliliter to 10 milliliters of a substance when sized as described later. For one exemplary syringe system, barrel portion (14) is further mechanically associated with a plunger (18). Said plunger typically comprises a piston connected to a first end of a shaft and wherein the piston is disposed inside barrel portion (14) with the shaft extending outside barrel portion (14) to a second end associated with a thumb rest (20). Such a syringe system typically includes opposed finger flanges (16) disposed at some point along the outside of barrel (14). The free end (22) of said barrel is ether associated with a needle/cannulas or an access port (configured to receive a conduit such a rubber tube).

### Apparatus Body

Referring now to FIG. 1 and FIG. 2, for the currently preferred embodiment, storage apparatus (10) comprises a body (24) that defines a generally regular polygonal outer perimeter. More specifically, body (24) defines a plurality of side-portions (30) that define a plurality of side-recesses (26). One embodiment of a side-recess (26) is a scallop as depicted in the various figures. Such scallops might not be considered a “polygonal” shape depending on one's view of how circles are formed. That said, for the purposes of this specification, the outer perimeter of body (24), as depicted in FIG. 1, defines a generally polygonal shape. It will be further appreciated that the plurality of side-portions (30), top surface (28) and said opposing bottom surface (29) define body-corners (34, FIG. 2) at the locations where all three features meet. Body-corners come in two types, normal body-corners (34) and recessed-body-corners (34r) and are collectively referred to as simply body-corners (34).

### Body-Corners

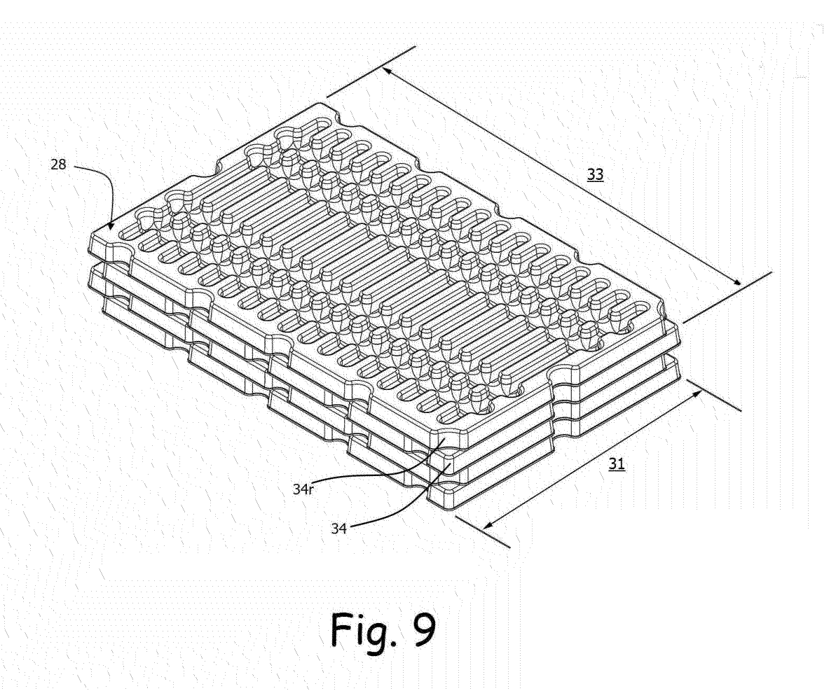

Preferably body (24) defines the same type body-corners at opposing corners as depicted in FIG. 2. In FIG. 2, body (24) defines two opposing normal body-corners (34) and two opposing recessed-body-corners (34r). Such helps identify the storage apparatus' (10) orientation relative to other trays when a plurality of trays are stacked on top of each other (see FIG. 9) and further provides a convenient location to grasp a tray among a stack of trays.

It will be appreciated that the number of body-corners defined by body (10) will generally determine the type of polygonal shape the perimeter of body (10) defines. Three corners will define a triangle, four corners will define a rectangle/square, five corners will define a pentagon, and so on. The body (24) defines at least three body-corners. For the preferred embodiment, however (as depicted in the various figures), the body (24) defines four body-corners with the perimeter defining a rectangle defining a body length (33) and a body width (31) (see FIG. 9).

### Top and Bottom Surfaces

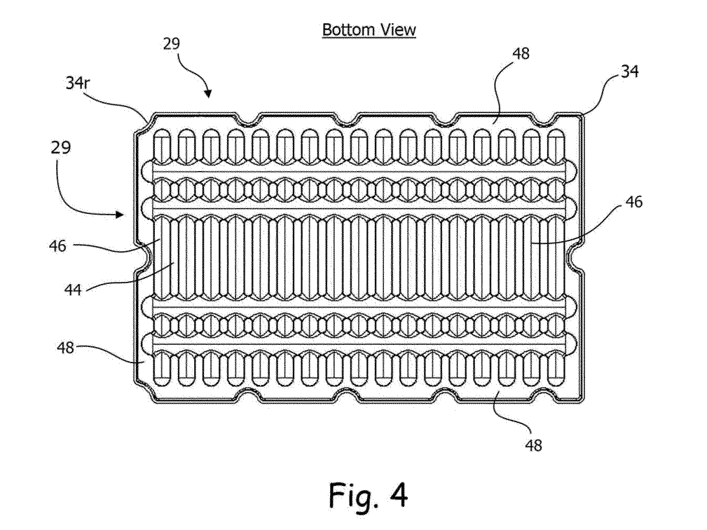

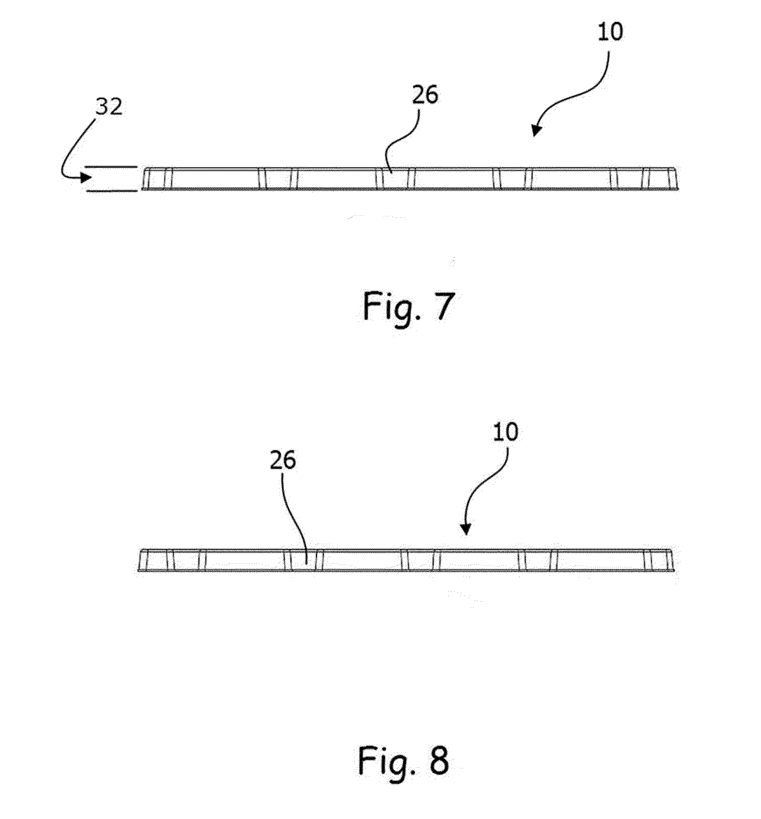

The body (24) comprises a top surface (28), an opposing bottom surface (29, FIG. 4), and a plurality of side-portions (30) defined along the perimeter of top surface (28) and opposing bottom surface (29). The side-portions (30) extend away from top surface (28) thereby defining a body height (32, FIG. 7) and/or depth.

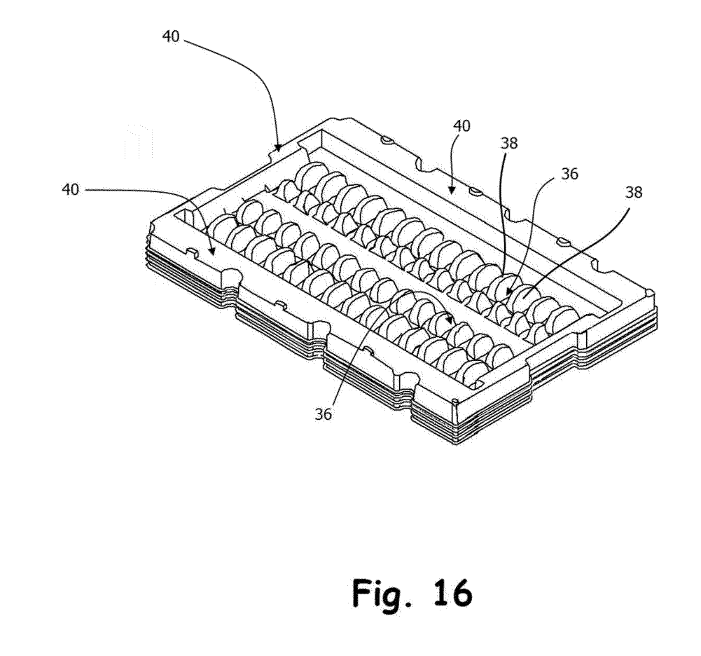

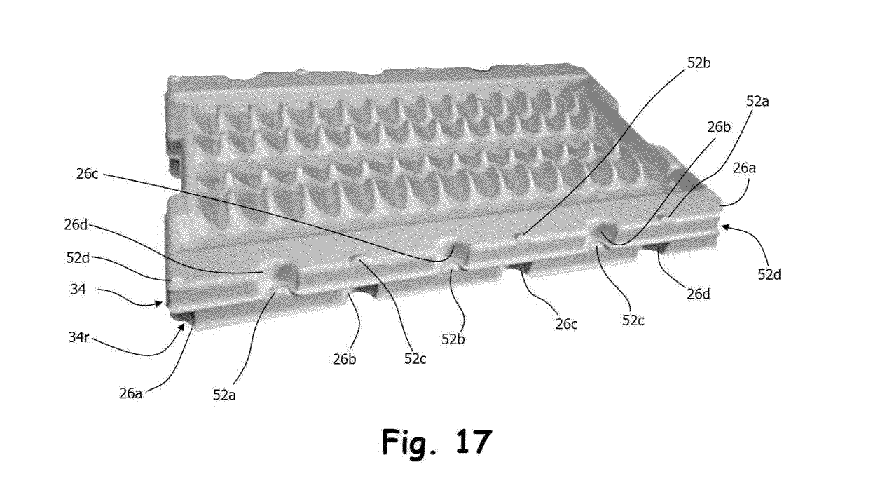

As can be seen in FIG. 1, top surface (28) is not a simple flat surface. Top surface (28) defines a plurality of ridges and channels. Notably, top surface (28) and bottom surface (29) define opposite surface features. Restated, where the top surface (28) defines a channel, the bottom surface will define a hollow channel ridge. Where the top surface defines a hollow ridge, the bottom surface will define a ridge void. Such a configuration allows nesting when pluralities of empty storage apparatuses are stacked on top of each other as depicted in FIG. 16.

Preferably, storage apparatus (10) is molded or thermoformed out of a heavy FDA approved ABS material to define a one piece structure. For one alternative embodiment, one or more side surfaces may be separate components that are mechanically associated with the top surface. Similarly, side-portions (30) may be configured to receive side-member extensions configured to increase the body height/depth. For such alternative configurations storage apparatus (10) can be configured to have various heights/depths by switching side-portions.

### Channels, Ridges, and Borders

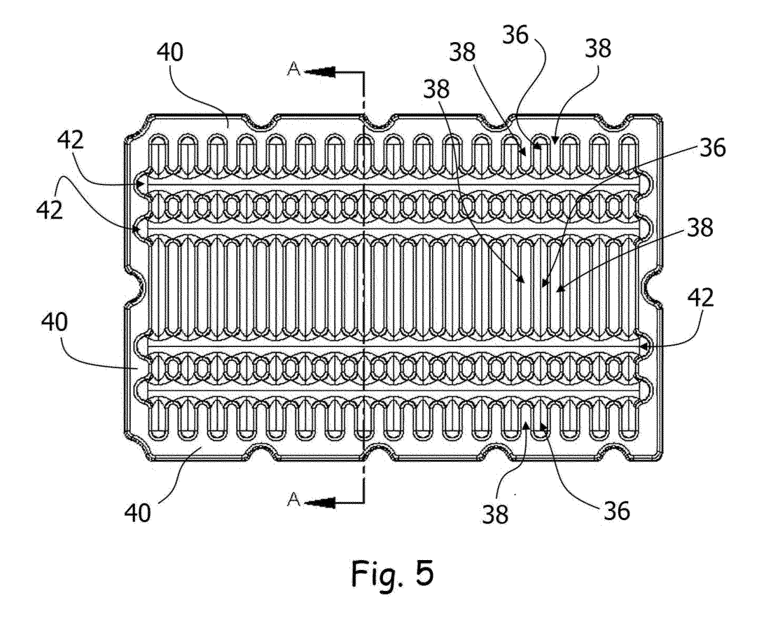

Referring now to FIG. 5, a top plan view of storage apparatus (10) is presented where the body (24), channels (36), and hollow ridges (38) are considered in more detail. The top surface (28) defines a plurality of channels (36) where adjacent channels are separated by a hollow ridge (38). The plurality of channels (36) and hollow ridges (38) are surrounded by a hollow top border region (40). For the current embodiment, the hollow top border region (40) is flat.

It should be appreciated that the term “hollow top border region” and “hollow ridges” means that such border/ridge does not extend the full depth of the storage apparatus (10) (i.e. the opposing side defines a void). Similarly, a “hollow ridge” is not a solid ridge that extends the full depth of the storage apparatus.

Notably, for the purposes of this document, for the preferred embodiments, channels (36) extend from the border region (40) on one side of body (24) to the border region (40) on an opposing side of body (24) wherein such channels are parallel to a first side (30d in FIG. 2) and perpendicular to a second side (30b in FIG. 2). For the preferred embodiment, the hollow ridges (38) define at least one ridge gap (42, FIG. 5). For the exemplary embodiment depicted in FIG. 1 and as best seen in FIG. 5, for example, each hollow ridge (38) defines four ridge gaps (42). Notably the ridge gaps (42) defined by adjacent hollow ridges (38) are in alignment across top surface (28) thereby defining a cross channel relative to channels (36). It will be appreciated, however, that embodiments where such ridge gaps are not in alignment across the top surface fall within the scope and spirit of the invention. For example, the ridge gaps could define a staggered alignment. Further, embodiments where hollow ridges (38) define no ridge gaps fall within the scope and spirit of the invention and a top surface (28) may define a mixture of the two (ridges with and without gaps).

As best seen in FIG. 3, one purpose of the ridge gaps (42) is to provide a receiver configured to receive syringe features such as finger flanges (16) (note that the finger flanges are much wider than the syringe barrel). Another purpose is to provide an access point to barrel portion (14), for example, for easy removal of a syringe system being stored in storage apparatus (10). It will be further appreciated that ridge gaps (38) may be wide enough to allow the finger flanges of adjacent syringe systems to be positioned in the storage apparatus (10) in the same orientation (see FIG. 3). For example, if a finger flange is 0.125 inches wide, the ridge gap would be at least 0.250 inches wide. That said, any size ridge gaps may be used without departing from the scope and spirit of the invention.

### Bottom Surface

Referring now to FIG. 4, the bottom surface (29) is considered in more detail. FIG. 4 depicts a top plan view of the bottom surface (29) for the current exemplary embodiment of storage apparatus (10). Basically the bottom surface (29) should be considered the opposite of the top surface (28). More specifically, where the top surface (28) defines a hollow ridge (38), the bottom surface (29) defines a ridge-void (44). Restated, the ridge void defines the “hollow” portion of an opposing hollow ridge (38). Similarly, where the top surface (28) defines a channel (36) the bottom surface (29) defines a channel-bottom (46) and where the top surface defines a hollow top border region the bottom surface defines a border-void (48). Consequently, one of ordinary skill in the art will appreciated that when storage apparatus (10) is viewed from the bottom, a channel-bottom would appear to be a ridge, a ridge-void would appear to be a channel, and border-void (48) would appear to be a indention/trench surrounding the channels and ridges. More specifically, border-void (48) defines the hollow portion of the hollow top border region (40). Thus, opposing bottom surface (29) defines a channel bottom (46) for each channel (36), a ridge-void (44) for each hollow ridge, and a border-void for the hollow top border region (40).

### Side Portions

Attention is now directed to the side-portions (30) specifically depicted in FIG. 7 and FIG. 8. As noted earlier, the plurality of side-portions (30) and the top surface (28) and opposing bottom surface (29) define a body-corner (34) where such features meet. Consequently, for the preferred embodiment the number of body-corners (34) and side-portions (26) will be equal. For one alternative embodiment, where the perimeter of body (24) defines a rectangle, for example, body (24) may define only two opposing side-portions (30) or perhaps two opposing side portions and one end side-portion (30) thereby defining an open section. That said, for the preferred embodiment each side of top portion (28) is associated with a side-portion (30).

Further, as depicted in FIG. 7 and FIG. 8, the side-portions (30) extend away from the top surface (preferably the same direct and the distance) and past the bottom surface thereby defining a body-height (32). For one embodiment, each of the plurality of side-portions (26) extends away from said top surface at an angle greater (preferably slightly greater) than 90 degrees to enhance said nesting-storage configuration (see FIG. 16).

As depicted in the various figures, body (24) defines four side-portions (30a-30d, FIG. 2) which are referred to collectively as side-portions (30). More specifically, as best seen in FIG. 2, body (24) defines side-portion (30a) and opposing side-portion (30b) and side-portion (30c) and opposing side portion (30d). For the currently preferred embodiment, at least two opposing side-portions (30) each define a plurality of side-recesses (26).

### Side-Recesses

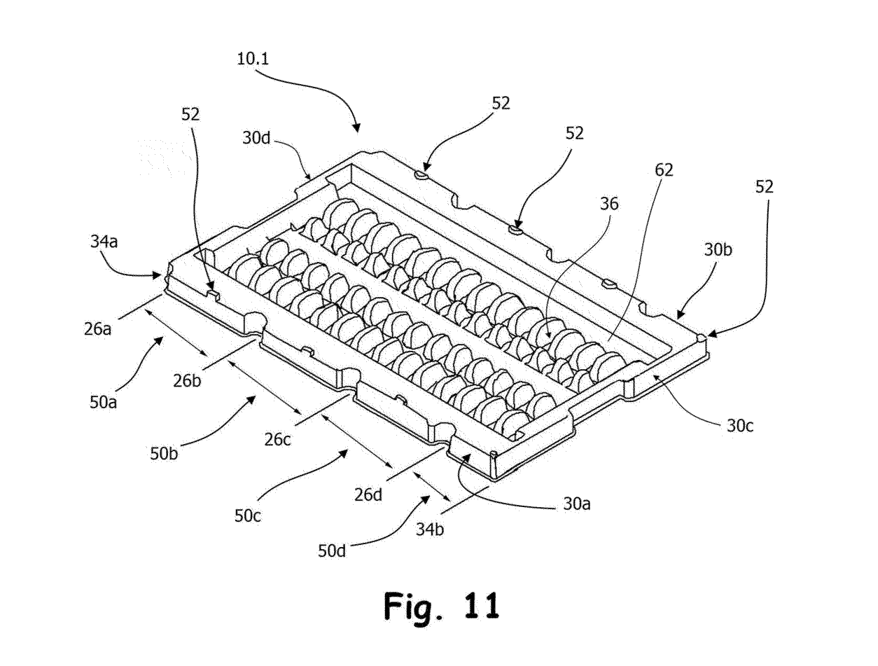

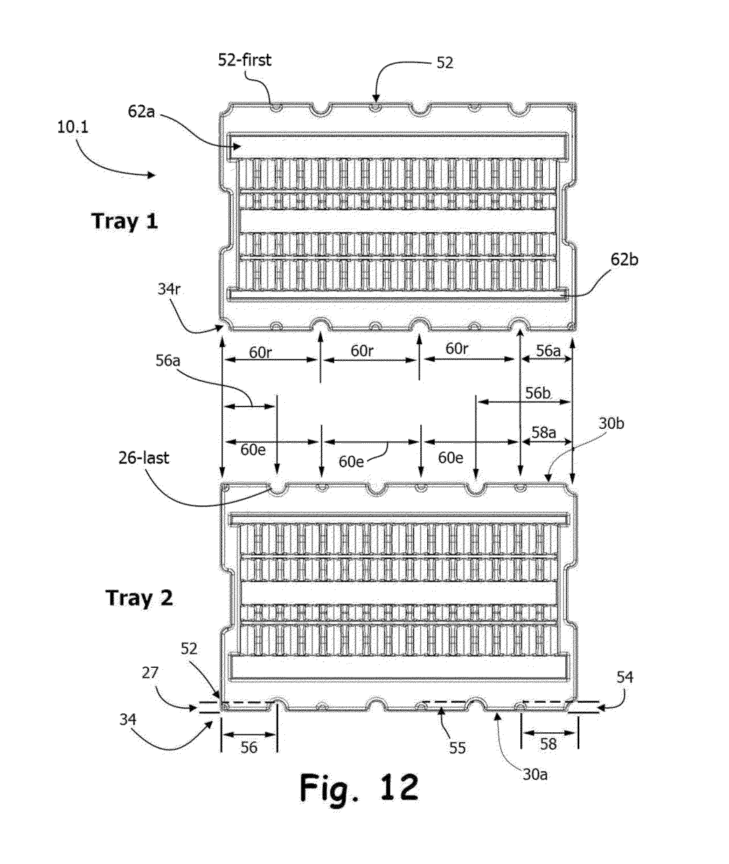

The alternative embodiment of storage apparatus (10) depicted in FIG. 11 and FIG. 12 will be used to discuss side-recesses (26). As depicted in FIG. 11, for the preferred embodiment the perimeter of side-recesses (26) define a semi-circle (“U” shape) or a scallop. Side-recesses (26) defining other shapes fall within the scope and sprite of the invention as well. For example, side-recesses (29) may define a “V” shape or an open box shape (a “square” missing a side). Whatever the shape, side-recess (26) define a recess-distance (27), FIG. 12) that extends inward relative to side-portion (30).

For the currently preferred embodiment as depicted in FIG. 11, the distance (50a) from a first side-recess (26b) and a first body-corner (34a) is different from the distance (50d) from a last side-recess (26d) to a second body-corner (34b). Such is true for both opposing side-portions (30a) and (30b). The distance between adjacent recesses is referred to as the recess-gap (50a, FIG. 11 or 60r, FIG. 12). For the embodiment presented in FIG. 11, recess-gap (50a) is substantially equal to recess-gap (50b) and (50C) but longer than last recess-gap (50d). One of ordinary skill in the art will appreciated that such a configuration will provide for a nesting-storage configuration (aka tray-storage mode) and a syringe-storage mode (aka stacking-storage mode) when two or more storage apparatuses (10) are stacked on top of each other (as explained in more detail below).

### Storage Modes

As depicted in FIG. 16, a tray-storage mode is defined as a nesting storage configuration. Such mode is particularly well suited for storing empty storage apparatuses (10). For such mode, a plurality of storage apparatuses (10) are stacked on top of each other so that the upper storage apparatus slides over the lower storage apparatus and the lower storage apparatus slides up into the upper storage apparatus thereby reducing the vertical height (and volume) required to store such configuration of storage apparatuses (10). Such tray-storage mode is accomplished by orienting/aligning the various storage apparatuses (10) in the same orientation so that the side-recesses of a lower storage apparatus (10) align with the side-recesses of an upper storage apparatus (10). Again, the tray-storage mode would typically be used to store a plurality of storage apparatuses (10) when empty as depicted in FIG. 16.

Referring now to FIG. 9 and FIG. 17, a syringe-storage mode is depicted showing a stacking storage configuration. In the syringe-storage mode a plurality of storage apparatuses (10) are stacked on top of each other so that the upper storage apparatus does NOT slide over the lower storage apparatus and the lower storage apparatus does NOT slide up into the upper storage apparatus. Instead, the upper storage apparatus sits on top of the lower storage apparatus. Such syringe-storage mode is accomplished by orientating an upper storage apparatus out of alignment (typically 180 degrees) compared to a lower storage apparatus so that the side-recesses do not align. When not in alignment, the side recess of an upper storage apparatus will rest on the top border region of the lower storage apparatus. The syringe-storage mode is ideal for use when pluralities of storage apparatuses (10) are associated with syringe systems (12, FIG. 3).

Notably, one of ordinary skill in the art will appreciate that it is preferable for each of the channels (36) to be configured to receive a syringe system (12) so that the upper most portion of the syringe system (12) is lower than the upper most portion of said top surface (28) when the syringe system (12) is associated with a channel. Such a configuration will prevent the bottom surface of an upper storage apparatus from touching the syringe system (12) associated with a lower storage apparatus when a plurality of storage apparatuses are configured in syringe-storage mode.

### Vertical Extensions

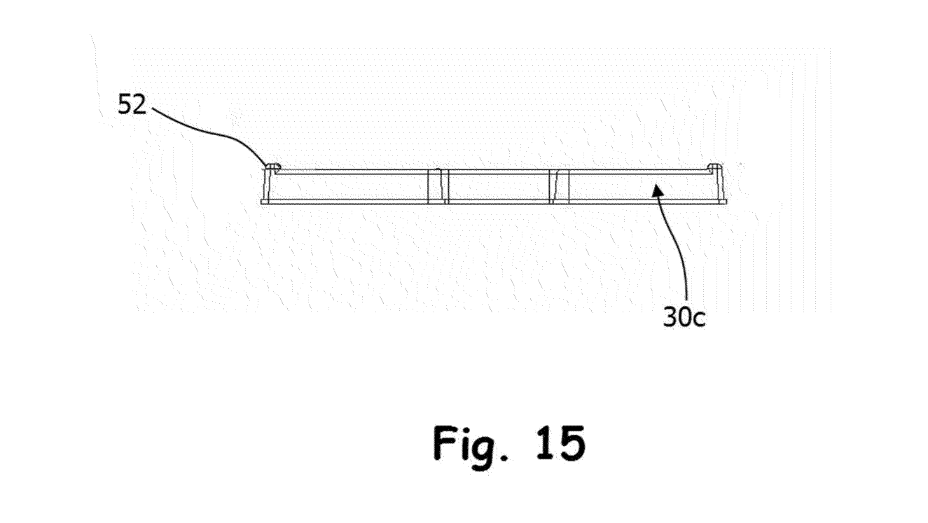

Referring now to FIG. 11-FIG. 17, the vertical extension feature is considered in more detail. Exemplary embodiments for such vertical extensions (52) include nubs, expansions, bulges and any other suitable surface feature that will associate with recesses to retard movement as describe below. Ideally, the shape of the vertical extensions is similar to the shape of the side-recesses. For example, if the side-recess defines a “V” shape, the vertical extensions would define a pie wedge shape that mates with the side-recess “V” shape. Notably, it will be appreciated that vertical extensions (52) are optional as shown in FIG. 1-FIG. 10.

For clarity, there are now two identically shaped storage apparatuses considered (as depicted in FIG. 12). Not that the two storage apparatuses are 180 degrees out of alignment in FIG. 12. When stacked on top of each other in one of the previously described tray-storage-mode (FIG. 16) or the syringe-storage-mode (FIG. 17), one storage apparatus will be referred to as the bottom storage apparatus and the other will be referred to as the top storage apparatus.

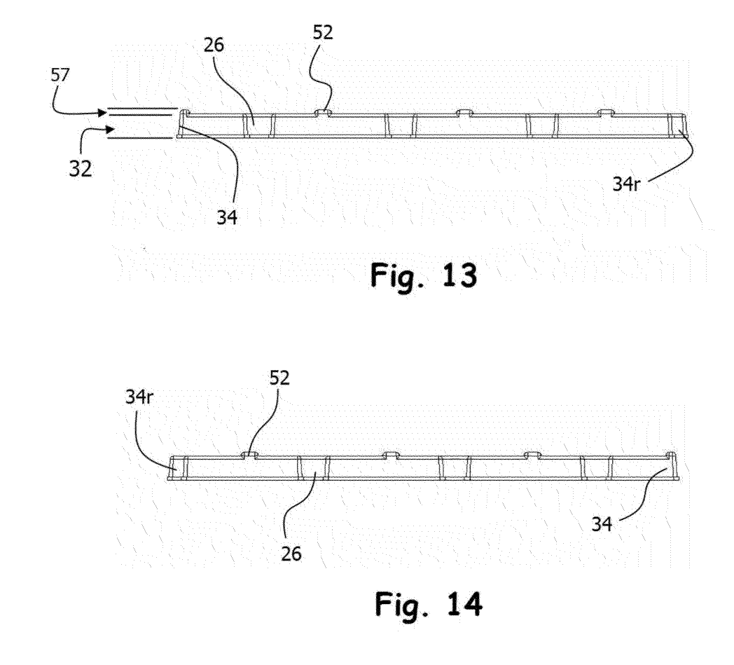

For one exemplary embodiment, the top surface (28) further defines a vertical-extension (52) disposed along its edge and between adjacent side-recesses (26). Preferably such vertical-extension(s) (52) extend vertically upward a predefined distance to define an extension-height (57, FIG. 13) and a predefined distance inward to define an extension-inset-distance (54, FIG. 12). Ideally the extension-height (57) would not be greater than the body-height (32) and the extension-insert-distance (54) is slightly less than recess-distance (27) defined by the side-recesses (26).

As best seen in FIG. 12, for the preferred embodiment, the relative distances of the side-recesses (26) and the vertical-extensions (52) are considered. Again, it should be appreciated that FIG. 12 presents two storage apparatuses (10) (called Tray 1 and Tray 2) orientated 180 degrees out of alignment relative to each other as indicated by the body-corners (34) and (34r). When Tray 2 is set on top of Tray 1 as depicted in FIG. 17, the vertical extensions (52) will be associated with body-recesses (26) and a body-corner (34r) and will impede if not prevent horizontal movement (movement along the X-Y axis) between Tray 1 and Tray 2.

Opposing side-portion (30a) and (30b) each define four side-recesses (26) and four vertical-extensions (52). The distance between adjacent side-recess (26) defines the recess-gap (60r) and the distance between adjacent vertical-extensions (52) defines the extension-gap (60e). For the preferred embodiment, the recess-gap (60r) and the extension-gap (60e) are substantially equal. However, the first side-recess (26) and the first vertical-extension (52) start a different points along side-portions (30) thereby causing the side-recess (26) to align with vertical-extension (52) when the two trays are 180 out of alignment (or out of “phase” if you will) as depicted in FIG. 12.

The distance (56a) from a last side-recess (26) to the nearest body-corner (34) defines the last side-recess location. Similarly, the distance (58a) from an opposing body-corner (34r) to a first side-recess (26) defines the first side-recess location. As best seen in FIG. 12, distance (56b) is longer than distance (56a). Similarly, the distance (58a) from an opposing body-corner (34r) to a first vertical-extension (52) defines the first vertical-extension location. Notably, distance (58a) is substantially equally to distance (56a) so that the first vertical-extension (52—first, of Tray 1) lines up with the last side-recess (26—last, of Tray 2).

Please note that for this example, when counting side-recesses, corner recesses (i.e. 34r) are counted. Consequently, as best seen in FIG. 17, when Tray 2 is stacked on top of Tray 1, a first side-recess (26a, Tray 2—top tray) is in alignment with a last vertical extension (52d, Tray 1, lower tray). The remaining side-recesses (26) and vertical-extensions (52) align is a similar manner. Stated differently, the first vertical extension (52a) defined by bottom Tray 1 is now associated (in vertical and horizontal alignment) with the last side-Recess (26d) defined by Tray 2. The second vertical-extension (52b) define by bottom Tray 1 is now associated with side-recess (26c) defined by upper Tray 2. The third vertical-extension (52c) defined by bottom Tray 1 is now associated with side-recess (26b) defined by upper Tray 2. Finally, the last vertical-extension (52d) defined by bottom Tray 1 is now associated with a first side-recess (26a). As noted above, such configuration defines a stacking-storage mode (syringe storage mode) that limits (if not prevents) horizontal movement between Tray 1 and Tray 2).

### Exemplary Measurements

For one preferred embodiment, exemplary storage apparatus measurements are as follows: (a) the body height (32, FIG. 13) is about 0.75 inches, (b) the extension height (57, FIG. 13) is about 0.25 inches above top surface (28), (c) the body width (31, FIG. 9) is about 12.0 inches, (d) the body length (33, FIG. 9), is about 18 inches, (e) the width of each channel (36, distance across the channel) is about 0.75 inches, (f) the width of each said hollow ridge (38) is about 0.32 inches, (g) the width of each said side-recess (at its widest point) is about 0.75 inches, (h) the distance between adjacent side-recesses, recess-gap (60r), is about 4.5 inches, and (i) the horizontal distance between a side-recess and a said vertical-extension is about 2.25 inches.

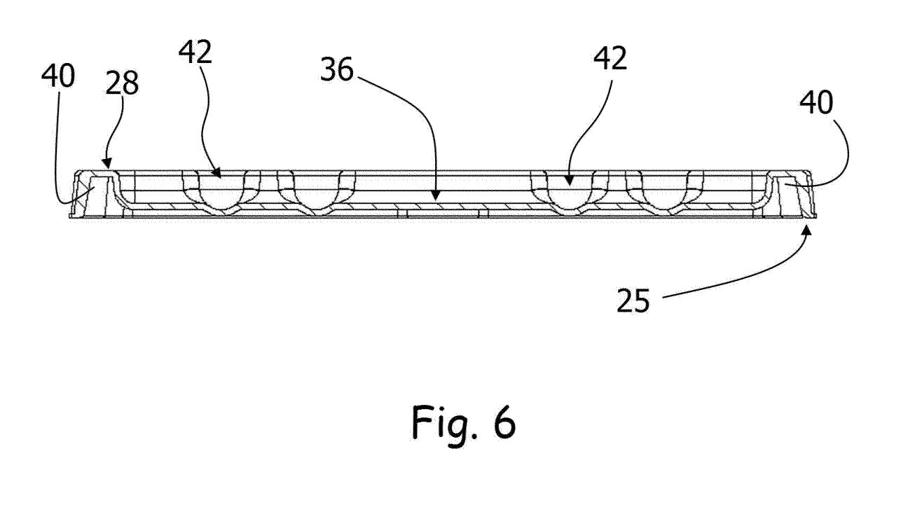

Additionally, as noted previously, storage apparatus (10) is preferably molded or thermoformed out of a heavy FDA approved ABS material (e.g. Acrylonitrile butadiene styrene (ABS)) to define a one piece structure so that said body thickness (25, FIG. 6) is about 0.120 to about 0.125 inches thereby making said storage apparatus crushproof in both said tray-storage mode and said syringe-storage mode for stacking amounts of 50 storage apparatuses or less.

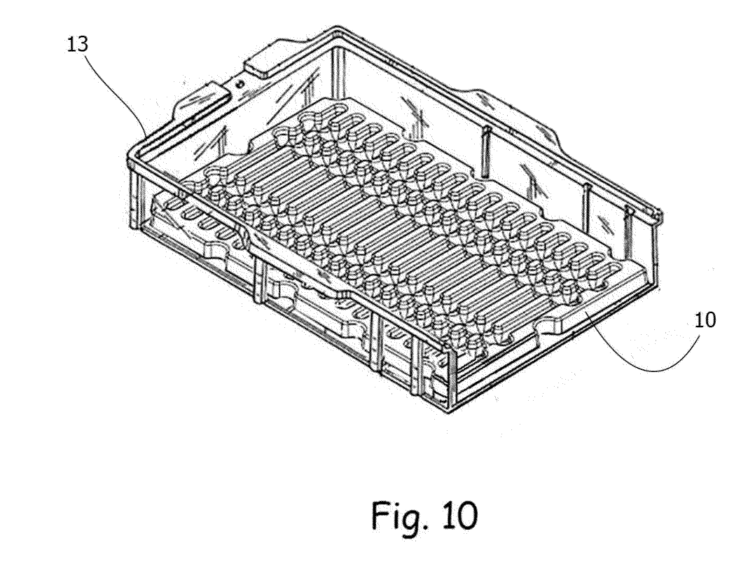

As depicted in FIG. 10, the above exemplary measurements make storage apparatus (10), comprising 15 channels, 14 hollow ridges, 8 side-recesses, and 6 vertical-extensions a perfect accessory for the tray storage system disclosed in commonly owned U.S. Pat. No. 7,766,164 issued to Hurst on 3 Aug. 2010.

### Alternative TOP and Bottom Surface Configuration

Referring now to FIG. 11, one alternative top surface (28) and opposing bottom surface (29) is presented. In FIG. 11, storage apparatus (10) defines a multi-mode storage tray (10.1) configured for storing a syringe system (12) (as before). For the currently preferred embodiment, body (24) defines a pair of opposed surfaces. Such pair of opposed surfaces comprise a top surface (28) and opposing bottom surface (29) where the distance between such opposed surfaces defines a body thickness (25, FIG. 6). The opposed surfaces further define a first side-portion (30a) defined along a first side of said top surface (28), a second side-portion (30b) defined along a second side of said top surface (28), a third side-portion (30c) defined along a third side of said top surface (28) and a fourth side-portion (30d) defined along a fourth side of said top surface (28). As before, such side portions (30) preferably extend away from top surface (28) about the same distance thereby defining a body depth (32) (or body height, both terms mean the same here).

As before, a first end of the first side-portion meets a first end of said second side portion to define a first body-corner (34). Similarly, a second end of said second side-portion (30) meets the first end of the third side portion (30) to define a second body-corner (34) and a second end of the third side-portion (30) meets the first end of the fourth side portion (30) to define a third body-corner (34). Finally, a second end of the forth side-portion meets the second end of the first side portion to define a forth body-corner (34).

As before, top surface (28) defines a plurality of channels (36) in horizontal alignment with at least one side-portion (30). Adjacent channels are separated by a hollow ridge (38).

A new feature relates to the configuration where the plurality of channels (36) and hollow ridges (38) are surrounded by an open-top-border-region (62) and such open-top-border-region (62) is further surrounded by a hollow-top-border-region (40). Thus, an open-top-border-region has been added. Notably opposing side portions (30a) and (30b) defined open-top-border-regions (62a) and 62b) where such regions have different widths. It will be appreciated that such regions could define the same width without departing from the scope and spirit of the invention.

As before, the opposing bottom surface (29) defines a channel bottom surface for each said channel and a ridge-void that defines the hollow portion of each hollow ridge. Bottom surface (29) further defines a border-void-region defining the hollow portion of said hollow-top-border region. Bottom surface defines a flat zone for the area opposing the open-top-border-regions (62).

As before, at least two opposing side-portions (30) each define a plurality of side-recesses (26). The distance from a first side-recess (26) to a first body-corner (34) is different from the distance from a last side-recess (26) to a second body-corner (34). Such is true for both of the two opposing side-portions. As before, such a configuration provides a tray-storage mode (nesting configuration) and a syringe-storage mode when the body of a first multi-mode storage tray (10.1) is associated with a second multi-mode storage tray (10.1).

Each of the channels (36) are configured to receive a syringe system so that the upper most portion of the syringe system (12) is lower than the upper most portion of said top surface (28) when said syringe system (12) is associated with a channel (36).

For one configuration, the multi-mode storage tray (10.1) comprises a top surface (28) that further defines a plurality vertical-extension (52) disposed between adjacent side-recesses (26) so that the vertical-extensions (52) associate with a side-recess (26) defined by a second multi-mode storage tray (10.1) when the two trays are stacked on top of each other and oriented in a stacking-storage mode as depicted in FIG. 17.

### Storage Apparatus Cover

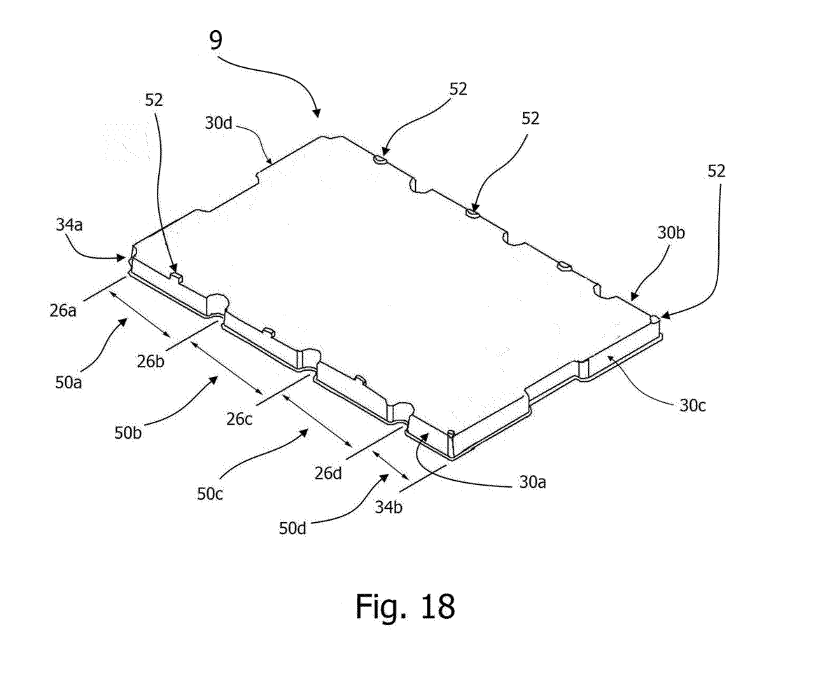

Referring now to FIG. 18, one exemplary embodiment of a storage apparatus cover (9) is presented. For the currently preferred embodiment, storage apparatus cover (9) is identical to a storage apparatus (10) except that the top surface (28) and the bottom surface (29) define a substantially flat surface. Thus, such a storage apparatus cover (9) would be used to cover one storage apparatus (10).

or one alternative embodiment, the height of storage apparatus cover (9) may be multiples of the height of storage apparatus (10). For example, if the body-height of storage apparatus (10) is 0.75 inches, the height/depth of storage apparatus cover (9) could be slightly more than 7.5 inches (or 10 times the height of a storage apparatus (10)). Such a cover (9) configuration could be used to cover and protect a stack of 10 storage apparatuses (10). And a plurality of such 10 stack configurations could be stacked on top of one another so that 50 storage apparatuses would be stacked on top of each other with each 10 being covered by a cover (9) (i.e. 5 covers). Such a configuration also adds structural integrity to the stack and makes inventory counting much quicker.

One of ordinary skill in the art will further appreciate that any shape or side cover (9) could be provided without departing from the scope and spirit of the invention. For example, cover (9) could be just over 2 times a wide as a storage apparatus (10) and just over 5 times as high as storage apparatus (10). Such a cover (9) would be ideal for covering two side-by-side stacks of 5 storages apparatuses (10).

Storage apparatus cover (9) may provide further features such as light blocking. Cover (9) may be constructed from a material that blocks all light. Alternatively, the cover (9) may be constructed of materials that provide wave-length specific blocking properties and such cover may or may not be transparent.

While the present subject matter has been described in detail with respect to specific embodiments thereof, it will be appreciated by those skilled in the art, upon attaining an understanding of the foregoing may readily adapt the present technology for alterations to, variations of, and equivalents to such embodiments. Accordingly, the scope of the present disclosure is by way of example rather than by way of limitation, and the subject disclosure does not preclude inclusion of such modifications, variations, and/or additions to the present subject matter as would be readily apparent to one of ordinary skill in the art.

1. A storage apparatus configured for storing a syringe system, said storage apparatus comprising:
a body having a generally polygonal outer perimeter, said body comprising a top surface, an opposing bottom surface, and a plurality of side-portions defined along the perimeter of said top surface and said opposing bottom surface wherein at least two opposing side-portions extend away from said top surface thereby defining a body-height;
wherein said top surface and said opposing bottom surface define at least three body-corners;
wherein said top surface defines a plurality of channels wherein adjacent channels are separated by a hollow ridge wherein said plurality of channels and hollow ridges are surrounded by a hollow top border region;
wherein each of said channels is configured to receive a syringe system so that the upper most portion of the syringe system is lower than the upper most portion of said top surface when said syringe system is associated with a channel; and
wherein two opposing side-portions each define a plurality of side-recesses wherein the distance from a non-corner first side-recess to a first body-corner is different from the distance from a last side-recess to a second body-corner thereby providing for a nesting-storage configuration and a stacking-storage configuration.

2. A storage apparatus configured for storing a syringe system as in claim 1, wherein said body defines a rectangular tray defining four side-portions wherein each side-portion runs the full length of the perimeter of a side of said top surface so that said four side-portions, said top surface and said opposing bottom surface define a body-corner where they meet thereby defining four body-corners;

3. A storage apparatus configured for storing a syringe system as in claim 2, wherein the side-recesses defined by said two opposing side-portions are positioned at the same location along their perspective side-portion.

4. A storage apparatus configured for storing a syringe system as in claim 3, wherein each of said plurality of side-portions extend away from said top surface at an angle greater than 90 degrees to enhance said nesting-storage configuration.

5. A storage apparatus configured for storing a syringe system as in claim 3, wherein said top surface further defines a vertical-extension disposed between adjacent side-recesses and suitably positioned to provide a horizontal movement limiting function when a plurality of storage apparatuses are oriented in said stacking-storage configuration.

6. A storage apparatus configured for storing a syringe system as in claim 1, wherein each said plurality of channels define a barrel-section configured to hold at least part of the barrel of said syringe system and a plunger-section configured to hold the plunger of said syringe system and a gap-section configured to allow gripping a portion of said syringe system.

7. A storage apparatus configured for storing a syringe system as in claim 5, wherein said tray is one piece formed from Acrylonitrile butadiene styrene (ABS) so that the distance from said top surface and said bottom surface is about 0.120 inches to about 0.125 inches thereby making said tray crushproof under normal use.

8. A multi-mode storage tray configured for storing a syringe system, said multi-mode storage tray comprising:
a body defining a pair of opposed surfaces, said pair of opposed surfaces comprising a top surface and opposing bottom surface wherein the distance between said opposed surfaces defines a body thickness;
wherein said opposed surfaces further define a first side-portion defined along a first side of said top surface, a second side-portion defined along a second side of said top surface, a third side-portion defined along a third side of said top surface, a fourth side-portion defined along a fourth side of said top surface;
wherein said first side-portion, said second side-portion, said third side-portion, and said fourth side-portion extend away from said top surface about the same distance in the same direction thereby defining a body height;
wherein a first end of said first side-portion meets a first end of said second side portion to define a first body-corner and wherein a second end of said second side-portion meets the first end of said third side portion to define a second body-corner and wherein a second end of said third side-portion meets the first end of said fourth side portion to define a third body-corner and wherein a second end of said forth side-portion meets the second end of said first side portion to define a forth body-corner
wherein said top surface defines a plurality of channels in horizontal alignment with at least one side-portion and wherein adjacent channels are separated by a hollow ridge wherein said plurality of channels and hollow ridges are surrounded by a hollow top border region;
wherein at least two opposing side-portions each define a plurality of side-recesses wherein the distance from a first side-recess to a first body-corner is different from the distance from a last side-recess to a second body-corner for both of said two opposing side-portions thereby providing for a tray-storage mode and a syringe-storage mode;
wherein said top surface further defines a vertical-extension disposed between adjacent side-recesses; and
wherein each of said channels is configured to receive a syringe system so that the upper most portion of the syringe system is lower than the upper most portion of said top surface when said syringe system is associated with a channel.

9. A multi-mode storage tray as in claim 8, wherein a side-recess is defined at said first body-corner and said second body-corner.

10. A multi-mode storage tray as in claim 9, wherein each of said side-portion extends away from said top surface at an angle greater than 90 degrees to enhance said tray-storage mode.

11. A multi-mode storage tray as in claim 8, wherein said vertical-extensions are suitably positioned to provide a horizontal movement limiting function when a plurality of storage apparatuses are stacked on top of each other in said syringe-storage mode.

12. A multi-mode storage tray as in claim 11, wherein each said channel defines a barrel-section configured to hold at least part of the barrel of said syringe system and a plunger-section configured to hold the plunger of said syringe system and a gap-section configured to allow gripping a portion of said syringe system.

13. A multi-mode storage tray as in claim 12, wherein said tray is one piece formed from Acrylonitrile butadiene styrene (ABS) so that said body thickness is about 0.120 to about 0.125 inches thereby making said tray crushproof in both said tray-storage mode and said syringe-storage mode for stacking amounts of 50 trays or less.

14. A multi-mode storage tray as in claim 13, wherein said tray height is about 0.75 inches, the height of each said vertical-extension is about 0.25 inches above said top surface, the width of said tray is about 12.0 inches, the length of said tray is about 18 inches, the width of each said channel is about 0.75 inches, the width of each said hollow ridge is about 0.32 inches, the width of each said side-recess is about 0.75 inches, the distance between adjacent side-recesses is about 4.5 inches, and the distance between a side-recess and a said vertical-extension is about 2.25 inches.

15. A multi-mode storage tray as in claim 14, wherein said tray comprises 15 channels, 14 hollow ridges, 8 side-recesses, and 6 vertical-extensions.

16. A multi-mode storage tray as in claim 14, wherein said vertical-extensions from the complementary shape relative to said side-recesses.

17. A multi-mode storage tray configured for storing a syringe system, said multi-mode storage tray comprising:
a body defining a pair of opposed surfaces, said pair of opposed surfaces comprising a top surface and opposing bottom surface wherein the distance between said opposed surfaces defines a body thickness;
wherein said opposed surfaces further define a first side-portion defined along a first side of said top surface, a second side-portion defined along a second side of said top surface, a third side-portion defined along a third side of said top surface, a fourth side-portion defined along a fourth side of said top surface;
wherein said first side-portion, said second side-portion, said third side-portion, and said fourth side-portion extend away from said top surface about the same distance thereby defining a body height;
wherein a first end of said first side-portion meets a first end of said second side portion to define a first body-corner and wherein a second end of said second side-portion meets the first end of said third side portion to define a second body-corner and wherein a second end of said third side-portion meets the first end of said fourth side portion to define a third body-corner and wherein a second end of said forth side-portion meets the second end of said first side portion to define a forth body-corner
wherein said top surface defines a plurality of channels in horizontal alignment with at least one side-portion and wherein adjacent channels are separated by a hollow ridge wherein said plurality of channels and hollow ridges are surrounded by an open-top-border-region and wherein said open-top-border-region is surrounded by a hollow-top-border-region and;
wherein said opposing bottom surface defines a channel bottom surface for each said channel and wherein said opposing bottom surface further defines a ridge-void defining the hollow portion of each said hollow ridge, and wherein said opposing bottom surface further defines a border-void-region defining the hollow portion of said hollow-top-border region;
wherein at least two opposing side-portions each define a plurality of side-recesses wherein the distance from a first side-recess to a first body-corner is different from the distance from a last side-recess to a second body-corner for both of said two opposing side-portions thereby providing for a tray-storage mode and a syringe-storage mode when said body is associated with a second multi-mode storage tray; and
wherein each of said channels is configured to receive a syringe system so that the upper most portion of the syringe system is lower than the upper most portion of said top surface when said syringe system is associated with a channel.

18. A multi-mode storage tray as in claim 17, wherein a side-recess is defined at said first body-corner and said second body-corner of said two opposing side-portions and wherein each of said side-portion extends away from said top surface at an angle greater than 90 degrees to enhance said tray-storage mode.

19. A multi-mode storage tray as in claim 17, wherein said top surface further defines a vertical-extension disposed between adjacent side-recesses so that said vertical-extension associates with a side-recess of a second multi-mode storage tray when the two trays are stacked on top of each other and oriented in said tray-storage to retard sliding between trays.

20. A multi-mode storage tray as in claim 19, wherein said multi-mode storage tray defines a medical paraphernalia storage tray accessory wherein said multi-mode storage tray is suitably sized to be stacked inside said medical paraphernalia storage tray wherein said medical paraphernalia storage tray is about being about 18 inches long, about 12 inches wide and about 18 inches high and wherein said multi-mode storage tray height is about 0.75 inches, the height of each said vertical-extension is about 0.25 inches above said top surface, the width of said multi-mode storage tray is about 11.5 inches, the length of said multi-mode storage tray is about 18 inches, the width of each said channel is about 0.75 inches, the width of each said hollow ridge is about 0.32 inches, the width of each said side-recess is about 0.75 inches, the distance between adjacent side-recesses is about 4.5 inches, and the distance between a side-recess and a said vertical-extension is about 2.25 inches and wherein said multi-mode storage tray is one piece formed from Acrylonitrile butadiene styrene (ABS) so that said body thickness is about 0.120 inches and 0.125 inches thereby making said multi-mode storage tray crushproof in both said tray-storage mode and said syringe-storage mode for stacking amounts of 50 multi-mode storage tray or less.
